(19) United States
(12) Patent Application Publication
(10) Pub. No.: US 2026/0060822 A1
(43) Pub. Date: Mar. 5, 2026

(54) BIOENGINEERED SCAFFOLDS FOR VASCULAR GRAFTS AND METHODS OF USE THEREOF

(71) Applicant: WAKE FOREST UNIVERSITY HEALTH SCIENCES, Winston-Salem, NC (US)

(72) Inventors: Jeko MADJAROV, Winston-Salem, NC (US); Sofia Jekova MADJAROVA, Winston-Salem, NC (US)

(21) Appl. No.: 19/105,924

(22) PCT Filed: Aug. 24, 2023
(86) PCT No.: PCT/US2023/031050

§ 371 Date: Feb. 24, 2025

Related U.S. Application Data
(60) Provisional application No. 63/400,599, filed on Aug. 24, 2022.

(51) Int. Cl. A61F 2/848; A61F 2/82; A61L 27/04; A61L 27/06; A61L 27/18; A61L 27/24

(52) U.S. Cl. CPC A61F 2/848; A61L 27/042; A61L 27/06; A61L 27/18; A61L 27/24; A61F 2002/823; A61F 2220/0008

(57) ABSTRACT

Disclosed herein are devices having an inner core comprising a biomatrix; and an outer scaffold comprising a biocompatible polymer. Also disclosed herein are methods for supporting a blood vessel and/or for treating or preventing an aneurysm by disposing a device having an inner core comprising a biomatrix; and an outer scaffold comprising a biocompatible polymer on an outer surface of a blood vessel of a subject. The bioengineered scaffolds of the present disclosure can limit the progression of vessel dilation while maintaining the biochemical characteristics of the vessel wall. Furthermore, the disclosed devices provide a minimally invasive alternative to conventional graft replacement, making the device suitable for patients having comorbidities.

### BACKGROUND

Aneurysms of the ascending aorta (AAA), and the aorta in general, are recognized as a distinct process associated with the degradation of structural matrix proteins, particularly elastin and collagen, involving all layers of the vessel wall. Graft replacement for AAA is now performed relatively safely and remains an accepted surgical procedure. However, these procedures often carry high hospital mortality, particularly in aging and sickly population. A less invasive technique such as external aortic wrapping with vascular graft materials including DACRON is generally safe and often can be performed without cardiopulmonary bypass and circulatory arrest as an alternative to graft replacement.

However, current designs and materials that are used to control aortic dilatation have not been engineered for this purpose. In particular, these materials are stiff and the compliance mismatch causes strong fibrotic reactions and necrosis of the adventitia. The aorta is thus transformed into a passive, non-compliant conduit, which may induce damage to the aortic valve leaflets and potentially to the coronary arteries. This may additionally cause turbulence at both ends of the “treated” with DACRON aorta, which can lead to aneurysmal dilatation over time and further complicate the clinical status of the patient, as well as lead to additional interventions or patient's poor outcome, after a theoretically successful surgery.

### SUMMARY

In accordance with the purposes of the disclosed devices, methods as embodied and broadly described herein, the disclosed subject matter relates to bioengineered scaffolds for vascular grafts and methods of using thereof.

In some aspects, the device comprises an inner core comprising a biomatrix; and an outer scaffold comprising a biocompatible polymer.

In various aspects, the biocompatible polymer comprises polyetheretherketone (PEEK) or polyetherketoneketone (PEKK).

In some aspects, the outer scaffold further comprises a metal framework. The metal framework can include, for example, NITINOL® or stainless steel.

In some aspects, the device comprises a fastening mechanism configured to secure placement of the device on an outer surface of a blood vessel In some aspects, the fastening mechanism comprises a zip and a lock.

In some aspects, the biomatrix is configured to allow migration of fibroblasts through the biomatrix. In some aspects, the biomatrix is collagen-enriched.

Additionally disclosed herein are methods of using the devices herein to treat or prevent an aneurysm by disposing the device on an outer surface of a blood vessel of a subject.

In some aspects, the blood vessel is an aortic blood vessel. In some aspects, the aortic blood vessel is an ascending aorta.

In some aspects, the outer scaffold maintains the size of the aorta over an extended time period.

In some aspects, the subject is an elderly subject.

In some aspects, the subject is not a candidate for aortic replacement.

In some aspects, the subject suffers from a connective tissue disorder. In some aspects, the connective tissue disorder is Marfan syndrome.

In some aspects, the method further includes fastening the device such that the biomatrix core contacts the adventitia of the blood vessel.

Also included herein are methods of supporting a blood vessel by disposing the device on an outer surface of the blood vessel of a subject.

In some aspects, the blood vessel is an aortic blood vessel. In some aspects, the aortic blood vessel is an ascending aorta.

In some aspects, the method further includes fastening the device such that the biomatrix core contacts the adventitia of the blood vessel.

The bioengineered scaffolds of the present disclosure can limit the progression of vessel dilation while restoring the biochemical characteristics of the vessel wall.

Moreover, the disclosed scaffolds exhibit strong compliance with the vessel wall, thereby increasing in vivo stability.

In addition, the disclosed devices provide a minimally invasive alternative to conventional graft replacement, making the device suitable for patients having comorbidities.

Additional advantages of the disclosed devices and methods will be set forth in part in the description which follows. The advantages of the disclosed devices and methods will be realized and attained by means of the elements and combinations particularly pointed out in the appended claims. It is to be understood that both the foregoing general description and the following detailed description are exemplary and explanatory only and are not restrictive of the disclosed devices and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

The accompanying figures, which are incorporated in and constitute a part of this specification, illustrate several aspects of the disclosure, and together with the description, serve to explain the principles of the disclosure.

FIGS. 1-2 depict an embodiment of the bioengineered scaffold having a type of zip and lock fastening mechanism.

FIG. 3 shows the device disposed on the adventitia of the ascending aorta to restrict vessel dilation according to one aspect of the disclosure.

### DETAILED DESCRIPTION

The devices and methods described herein may be understood more readily by reference to the following detailed description of specific aspects of the disclosed subject matter and the Examples included therein.

Before the present devices and methods are disclosed and described, it is to be understood that the aspects described below are not limited to specific methods devices, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Values can be expressed herein as an “average” value. “Average” generally refers to the statistical mean value.

By “substantially” is meant within 5%, e.g., within 4%, 3%, 2%, or 1%. “Exemplary” means “an example of” and is not intended to convey an indication of a preferred or ideal embodiment. “Such as” is not used in a restrictive sense, but for explanatory purposes.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included. The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

The terms “treat,” “treating,” “treatment,” “therapy”, and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments may be applied preventively, prophylactically, palliatively or remedially. Treatments are administered to a subject prior to onset (e.g., before obvious signs of aneurysms), during early onset (e.g., upon initial signs and symptoms of aneurysms), or after an established development of aneurysms. Prophylactic administration can occur for several days to years prior to the manifestation of symptoms of an infection.

As used herein, by a “subject” is meant an individual. Thus, the “subject” can include domesticated animals (e.g., cats, dogs, etc.), livestock (e.g., cattle, horses, pigs, chickens, ducks, geese, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. As used herein, “elderly subject” refers to a human subject, generally over 60 years of age, and more typically over 65 years of age, such as over 70 years old.

The term “aortic aneurysm” as used herein is intended to relate to and include thoracic aneurysms, abdominal aneurysms, and related vessel diseases. As used herein, the term “blood vessel” is defined as a vessel or canal through which blood circulates. Examples include an artery, a vein, and/or a capillary.

The term “connective tissue disorder” refers to diseases and conditions that affect connective tissue, specifically collagen. In some examples, the connective tissue disorder includes Marfan's Syndrome, Loeys-Dietz Syndrome, Familial Aortic Aneurysm, Bicuspid Aortic Valve with Aortic Dilation, Mitral Valve Prolapse Syndrome, Marfan Habitus, Congenital Contractural Arachnodactyly (Beals Syndrome), Sjögren's syndrome, Stickler syndrome, Shprintzen-Goldberg syndrome, Weill-Marchesani syndrome, and/or Ehlers-Danlos syndrome. In some examples, the device is used to support a blood vessel in a patient having Marfan's Syndrome.

As used herein, “biomatrix” refers to an isolated soft tissue extract enriched in extracellular matrix, as described herein, which retains many or most of the collagens and collagen-bound factors found naturally in the biological tissue. In some embodiments essentially all of the collagens and collagen-bound factors are retained and in other embodiments the biomatrix scaffold comprises all of the collagens known to be in the tissue.

The term “biocompatible,” as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

### Devices

Disclosed herein are devices including an inner core comprising a biomatrix; and an outer scaffold comprising a biocompatible polymer. In various implementations, the biocompatible polymer of the outer scaffold includes a polyarylethyl ketone (PAEK), such as polyether ether ether ketone (PEEEK), polyether ether ketone (PEEK), polyether ketone (PEK), polyether ether ketone ether ketone (PEEKEK), polyether ether ketone ketone (PEEKK), polyether ketone ketone (PEKK), polyether ketone ether ketone ketone (PEKEKK), copolymers thereof. For example, the biocompatible polymer can be polyetheretherketone (PEEK) and/or polyetherketoneketone (PEKK). In some other aspects, the biocompatible polymer includes a polymer such as polyethylene, polypropylene, polyurethane, polyglycolic acid, polyesters, polyamides, their mixtures, blends, copolymers, mixtures, blends and copolymers are suitable; preferred of this class are polypropylenes such as PROLENE or PROLENE mesh, polyesters such as polyethylene terephthalate including DACRON, ETHICON and MYLAR and poly aramids such as KEVLAR®, polyfluorocarbons such as polytetrafluoroethylene with and without copolymerized hexafluoropropylene (TEFLON or GORETEX), and porous or nonporous polyurethanes, and polyurethane ureas such as THORALON. Natural materials or materials based on natural sources such as collagen are also suitable.

In various aspects, the outer scaffold additionally includes a metal framework. For example, the metal framework can include a biocompatible material such as stainless steel, titanium alloys, tantalum alloys, nickel alloys such as nickel-chromium alloys, cobalt alloys such as cobalt-chromium alloys and precious metals. Shape memory alloys such as the nickel-titanium alloy, NITINOL® can also be used. In some aspects, the metal framework comprises NITINOL®) and/or stainless steel.

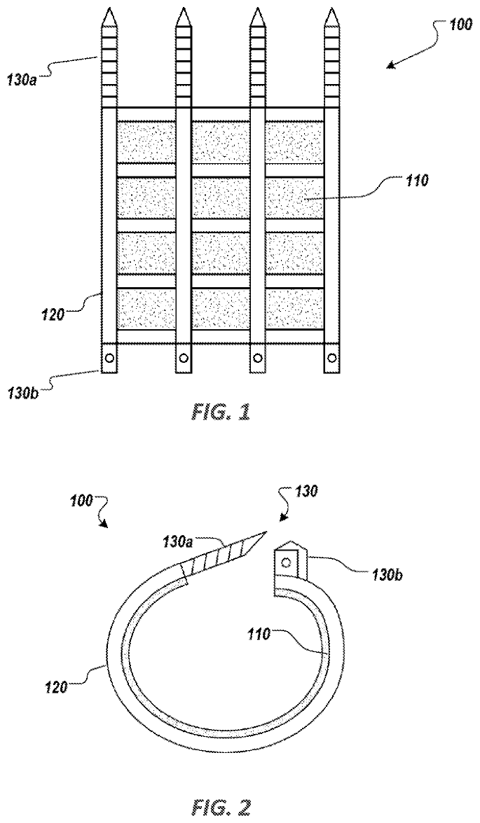

Various aspects include a fastening mechanism configured to secure the placement of the device on an outer surface of a blood vessel. For example, the fastening mechanism can include threads, screws, hooks, zip ties, fasteners, clips, flaps, claspers, springs, claspers, staplers, grips, zippers, hooks and corresponding eyes, hook and loop reclosable fastener squares, hook and loop reclosable fastener strips, hook and loop reclosable fastener dots, hooks-and-loops, e.g., Velcro™-type fasteners, straps, holes and string, sutures, wires, cables, tabs, poppers, nails, buttons and corresponding button holes, press buttons brackets, glues, adhesives, or any combination thereof. In some aspects, the fastening mechanism includes a zip and lock, such as the one shown in FIGS. 1-2. In other aspects, the fastening mechanism includes one or more zip ties.

In various aspects, the biomatrix is configured to allow the migration of fibroblast through the biomatrix. The term “fibroblast migration” or the like as used herein, refers to any movement of a fibroblast in the direction of tissue injury. Such migration is usually stimulated by chemotactic factors (i.e., for example, lysophosphatidic acid) released by white blood cells. For example, the biomatrix can include a porous material such that fibroblasts may freely traverse the biomatrix. In some embodiments, the biomatrix is collagen-enriched.

In various embodiments, the biomatrix comprises a material derived from a soft tissue extract. For example, the biomatrix can include an autologous tissue, homologous tissue, and/or heterologous tissue, such as tissue derived from organs or portions of organs, blood vessels, blood vessel-like tissue, heart, cardiac valves, pericardia, stomach, intestine, urinary bladder, dermis, and the like. For example, the heterologous tissue can include decellularized porcine aorta. In some embodiments, the biomatrix comprises an elastin rich tissue. In some embodiments, the biomatrix comprises a 3D printed biomatrix. In some embodiments, the biomatrix comprises a 3D printed collagen matrix.

In some aspects, the biomatrix can further include fibroblast migration mediators such as Growth Factors, Platelet derived growth factor (PDGF), Epidermal growth factor (EGF), also known as vascular endothelial, growth factor (VEGF); Macrophage derived growth factor (MDGF); Fibroblast growth factor (FGF); and Nerve growth factor (NGF). Blood Anti-Coagulation Proteins Antibodies or peptide fractions specific for any of blood Factors I-XII respectively; Antibodies or peptide fractions specific against Vitamin K; Hirudin; and Albumin. Selected cytokines (enzymes) Interleukin-1 (IL-1), an endogenous pyrogen and major inflammatory mediator; Interleukin-2 (IL-2), a T-cell activator and growth factor; Interleukin-3(IL-3), a hematopoietic growth factor; Interleukin-4 (IL-4), a T-cell and B-cell growth factor; Interleukin-5 (IL-5), a promoter of eosinophil growth and differentiation and IgA antibody synthesis; Interleukin-6 (IL-6), a B-cell differentiation factor; Interleukin-7 (IL-7), a growth factor for early B- and T-lymphocytes; Interleukin-8 (IL-8), a chemotactic factor for neutrophils and lymphocytes; Interleukin-10 (IL-10), a down-regulator of cell activation; Interleukin-12 (IL-12), an augmenter of IFN-γ production; Interleukin-13 (IL-13), a factor which overlaps in function with IL-4; Tumor necrotic factor (TNF), a factor which overlaps in function with IL-1 and mediates host response to gram-negative bacteria; Interferons -α, -β, -γ, which activate macrophages, enhance lymphocyte and natural killer cells, and have antiviral and antitumor activity; and Granulocyte-macrophage colony stimulating factor (GM-CSF), a growth factor for granulocytes, macrophages, and eosinophils Lectins (mitogenic agents from plants) Concanavalin A, a protein from the jack bean; UEA I, Glycoproteins and proteoglycans Ovalalbumin; and/or Avidin, Oligonucleotides, RGD (and proteins/peptides containing sequence); VCAM; ICAM; PCAM, Saccharides and polysaccharides; Glucosamine; Chondroitin and chondroitin 4-sulfate; Hyaluronic acid; and/or Heparin.

In some aspects, the device includes a therapeutic agent dispersed within the biocompatible polymer or biomatrix. In some examples, the therapeutic agent is dispersed inhomogeneously (e.g., randomly, along a concentration gradient, etc.). However, in other examples, the therapeutic agent is dispersed substantially homogeneously. The therapeutic agent can, for example, comprise an anti-inflammatory agent, analgesic agent, antimicrobial agent, or a combination thereof. As used herein, antimicrobials include, for example, antibacterials, antifungals, and antivirals.

In some examples, the therapeutic agent can comprise an anti-inflammatory agent, such as steroidal and/or non-steroidal anti-inflammatory agents. Examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Examples of non-steroidal anti-inflammatory drugs include acetaminophen, aspirin, ibuprofen, naproxen, Celebrex, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

In some examples, the therapeutic agent can comprise an analgesic. Examples of analgesics include, but are not limited to, 1-Iodomorphine; 3-Hydroxymorphinan; 4-Methylpregabalin; A-366,833; ABT-202; Aceburic acid; Acefurtiamine; Acetaminosalol; Acetyldihydrocodeine; Acetylmethadol; Adrenorphin; Alazocine; Algifen; Alimadol; Alletorphine; Alphacetylmethadol; Alphamethadol; Amidorphin; Aminophenazone; Ampyrone; Amrutanjan (balm); Anacin; Anadin; Analgecine; Anazocine; Anileridine; Anilopam; Anodyne; Askit Powders; Aspergum; Aspirin; Axomadol; AZD0328; BC Powder; Befiradol; Benorilate; Betacetylmethadol; Betahydroxy fentanyl; Betamethadol; Bicifadine; Biphalin; Brorphine; Bucetin; Bucinnazine; Butalbital; Butinazocine; Butonitazine; Butorphanol; Cannabidiol; Carbazocine; Cebranopadol; Chlorodyne; Chlorproethazine; Cinchophen; Cogazocine; Conolidine; Conorfone; CR-4056; CR665; Dasolampanel; Deltorphin; Deltorphin I; DepoDur; Desmetramadol; Desomorphine; Dezocine; Diacetylnalorphine; Dichloralphenazone; Difenamizole; Dimenoxadol; Dimepheptanol; Dimethylheptylpyran; Dinalbuphine sebacate; Dipipanone; Diproqualone; Dipyrocetyl; Dosulepin; DSP-2230; Embutramide; Enkephalinase inhibitor; Epibatidine; Epiboxidine; Eptazocine; Esterom; Etazocine; Ethylketazocine; Etodesnitazene; Etonitazepipne; Etonitazepyne; Etorphine; Famotidine; Faxeladol; Fedotozine; Fentanyl; Filenadol; Flumexadol; Flumizole; Fluproquazone; Frakefamide; Funapide; Gabapentin; Gabapentin enacarbil; Gabapentinoid; Glafenine; Homofentanyl; Homprenorphine; Ibazocine; Ibuprofen; Incarvillateine; Indantadol; Isomethadone; Isotonitazene; Isovaline; Kavalactone; Kelatorphan; Ketamine; Ketobemidone; Ketorfanol; Ketorolac; Lactucarium; Leconotide; Levallorphan; Levomepromazine; Levomethadone; Lufuradom; Magnesium alicylate; Mavatrep; Meconopsis horridula; Menabitan; Menthol; Menthoxypropanediol; Meprobamate; Meseclazone; Metacetamol; Metamizole; Methoxyflurane; Metkefamide; Metodesnitazene; Metonitazene; Mexiletine; Migraleve; Mirogabalin; Mitragyna speciosa; Moffett's solution; Moramide intermediate; Morpheridine; Morphiceptin; Morphine; Moxazocine; MP-2001; N-2′-IndolyInaltrexamine; Nabilone; Nafoxadol; Nalbuphine; Nalmexone; Naproxen; Nefopam; Nexeridine; NFEPP; Nimesulide; Noracymethadol; Norlevorphanol; Normethadone; Norpipanone; NS-11394; Oliceridine; Opiorphin; Opiranserin; Opium; Otenaproxesul; Oxilorphan; Paracetamol; Pethidine; PF-05089771; Phenacetin; Phenazone; derivatives thereof; and combinations thereof.

In some examples, the therapeutic agent can comprise an analgesic, such as an opioid. Examples of opioids include, but are not limited to, (α/β)-Meprodine; (α/β)-Prodine; 1-(4-Nitrophenylethyl)piperidylidene-2-(4-chlorophenyl)sulfonamide (W-18); 14-Cinnamoyloxycodeinone; 14-Ethoxymetopon; 14-Hydroxydihydrocodeine; 14-Hydroxymorphine; 14-Methoxymetopon; 14-Phenylpropoxymetopon; 18,19-Dehydrobuprenorphine (HS-599); 18-Methoxycoronaridine; 1-Bromocodeine; 1-Chlorocodeine; 1-Iodomorphine Codeine-6-glucuronide; 1-Nitrocodeine; 2,4-Dinitrophenylmorphine; 3-(3-Methoxyphenyl)-3-ethoxycarbonyltropane; 3-(dimethylamino)-2,2-dimethyl-1-phenylpropan-1-one; 3,14-Diacetyloxymorphone; 3,6-Dibutanoylmorphine; 3-Acetyloxymorphone; 3-Allylfentanyl; 3-Hydroxymorphinan; 3-Methylfentanyl; 3-Methylthiofentanyl; 3-Monoacetylmorphine; 4-Chlorophenylpyridomorphinan; 4-Fluoropethidine; 4-Phenylfentanyl; 5,6-Dihydronorsalutaridine; 5,9 alpha-diethyl-2-hydroxybenzomorphan (5,9-DEHB); 6-Acetyldihydromorphine; 6-Keto Nalbuphine; 6-Methyldihydromorphine; 6-Methylenedihydrodesoxy morphine; 6-Monoacetylcodeine; 6-Monoacetylmorphine; 6-Nicotinoyldihydromorphine; 7-Acetoxymitragynine; 7-Hydroxymitragynine; 7-PET; 7-Spiroindanyloxymorphone; 8,14-Dihydroxydihydromorphinone; 8-Carboxamidocyclazocine (8-CAC); Acetorphine; Acetoxyketobemidone; Acetylcodone; Acetyldihydrocodeine; Acetylmethadol; Acetylmorphone; Acetylpropionylmorphine; AD-1211; ADL-5859; AH-7921; Aknadinine; Akuammidine; Akuammine; Alazocine; Alfentanil; Alimadol; Alletorphine (N-allyl-noretorphine); AllyInorpethidine; Allylprodine; Alphaacetylmethadol; Alphamethadol; Alvimopan; Amentoflavone; Anazocine; Anileridine; Anilopam +HCl; Asimadoline; Axomadol; Azaprocin; AZD-2327; Azidomorphine; BDPC; Benzethidine; Benzhydrocodone; Benzylfentanyl; Benzylmorphine; Betacetylmethadol; Betamethadol; Bezitramide; Bisnortilidine; Bremazocine; Brifentanil; BRL-52537; Bromadol; Bromadoline; Bromocodide; Bromoisopropropyldihydromorphinone; Bromomorphide; BU-48; Buprenorphine; Buprenorphine-3-glucuronide; Butinazocine; Butorphanol; Butyrfentanyl; BW373U86; Carbazocine; Carfentanil; Carperidine; Cephakicine; Cephasamine; Chlornaltrexamine; Chlorodihydrocodide; Chloromorphide; Chloroxymorphamine; Ciprefadol; Ciramadol; Clonitazene; Codeine; Codeine methylbromide; Codeine-N-oxide; Codeine-N-oxide (genocodeine); Codeinone; Codide; Codoxime; Cogazocine; Conorfone (codorphone); Coronaridine; Cyclazocine; Cyclorphan; Cyprenorphine; Cyprodime; Cyproterone acetate; Desmethylclozapine; Desmethylmoramide; Desmethylprodine (MPPP); Desocodeine Desomorphine (dihydrodesoxymorphine); Dextromethadone; Dextromoramide; Dextropropoxyphene (propoxyphene); Dezocine; Diacetyldihydromorphine (dihydroheroin, acetylmorphinol); Diampromide; Dibenzoylmorphine; Dibutyrylmorphine; Diethylthiambutene; Difenoxin; Diformylmorphine; Dihydrocodeine; Dihydrocodeine; Dihydrodesoxy codeine (desocodeine); Dihydroetorphine; Dihydroisocodeine; Dihydromorphine; Dimenoxadol; Dimepheptanol (racemethadol); Dimethylmorphine (6-O-Methylcodeine); Dimethylthiambutene; Dioxaphetyl butyrate; Diphenoxylate; Dipipanone; Dipropanoylmorphine; Doxpicomine; DPI-221; DPI-287; DPI-3290; Drotebanol; Droxypropine; Embutramide; Enadoline; Eptazocine; Eseroline; Etazocine; Ethoheptazine; Ethyldihydromorphine; Ethylketazocine; Ethylmethylthiambutene; Ethylmorphine (dionine); Etonitazene; Etorphine; Etoxeridine (carbetidine); Faxeladol; FE 200665; Fedotozine; Fenfangjine G; Fentanyl; Fluorophen; Furethidine; Gemazocine; GR-89696; Herkinorin; Heroin (diacetylmorphine); Heroin-7,8-oxide; Heterocodeine; Hodgkinsine; Homprenorphine; Hydrocodone; Hydromorphinol; Hydromorphone; Hydroxycodeine; Hydroxypethidine (bemidone); HZ-2; Ibazocine; IBNtxA; Ibogaine; IC-26; ICI-199,441; ICI-204,448; Isoaminile; Isocodeine; Isomethadol; Isomethadone; Isotonitazene; Ketamine; Ketazocine; Ketobemidone; Ketorfanol; KNT-42; Kolokol-1; Lefetamine; Levacetylmethadol; Levargorphan; Levoisomethadone; Levomethadone; Levomethorphan; Levomoramide; Levophenacylmorphan; Levopropoxyphene; Levorphanol; Lofentanil; Loperamide; LPK-26; LS-115509; Lufuradom; Matrine; MCOPPB; Menthol; Meperidine-N-oxide; Meptazinol; Metazocine; Metethoheptazine; Methadone; Metheptazine; Methorphan (racemethorphan); Methyldesorphine; Methyldihydromorphine (dihydroheterocodeine); Methyldihydromorphinone; Methylketobemidone; Metofoline; Metonitazene; Metopon; Mirfentanil; Mitragynine; Mitragynine pseudoindoxyl; Morphanol (racemorphanol); Morphenol; Morpheridine; Morphine; Morphine methylbromide; Morphine-6-glucuronide; Morphine-N-oxide; Morphine-N-oxide (genomorphine); Morphinone; Morphol; Moxazocine; MT-45; MT-7716; Myrophine; Nalbuphine; Nalbuphone; Nalfurafine; Nalorphine; Nalorphine dinicotinate; Naltrexol; N-cyclopropylmethylnoretorphine; Nepenthone; Nexeridine; Nicocodeine; Nicodicodeine; Nicomorphine; N-Methylcarfentanil; N-Methylmorphinan; NNC 63-0532; Noracymethadol; Norbuprenorphine; Norbuprenorphine-3-glucuronide; Norcodeine; Noribogaine; Norlevorphanol; Normethadone; Normorphine; Noroxymorphone; Norpipanone; Norpropoxy phene; Nortilidine; N-Phenethyl-14-ethoxymetopon; N-Phenethyl-14-ethoxymetopon; N-Phenethylnordesomorphine; N-Phenethylnormorphine; Ocfentanil; O-Desmethyltramadol; Ohmefentanyl; Opium; Oripavine; Oxilorphan; Oxpheneridine (carbamethidine); Oxycodone; Oxymorphazone; Oxymorphol; Oxymorphone; Pantopon; Papaveretum (Omnopon); Parafluorofentanyl; Pentamorphone; Pentazocine; PEPAP; Pericine; Pethidine (meperidine); Phenadone; Phenadoxone (heptazone); Phenampromide; Phenaridine; Phenazocine; Phencyclidine; Pheneridine; Phenomorphan; Phenoperidine; Pholcodine (morpholinylethylmorphine); Picenadol; Piminodine; Piperidylthiambutene; Piritramide; Prodilidine; Profadol; Proglumide; Proheptazine; Properidine (ipropethidine); Propiram; Propylketobemidone; Prosidol; Proxorphan; Pseudoakuammigine; Pseudomorphine; Pyrrolidinylthiambutene; Pyrroliphene; PZM21; Quadazocine; R-30490; R-4066; Racemoramide; RAM-378; Remifentanil; Ro-1539; Ro4-1539; Ro64-6198; Ro65-6570; RWJ-394,674; Salvinorin A; Salvinorin B ethoxymethyl ether; Salvinorin B methoxymethyl ether; Sameridine; SB-612,111; SC-17599; Semorphone; SKF-10047; SNC-80; SoRI-9409; Spiradoline; SR-16435; SR-8993; Sufentanil; TAN-67; Tannagine; Tapentadol; Tetrapon; Thebacon; Thebacon (acetyldihydrocodeinone, dihydrocodeinone enol acetate); Thebaine; Thenylfentanyl; Thevinone; Thiambutene; Thiazocine; Thienorphine; Thiobromadol (C-8813); Thiofentanyl; Tifluadom; Tilidine; Tonazocine; Tramadol; Transisocodeine; Trefentanil; Trimebutine; Trimeperidine (promedol); U-47700; U-50,488; U-69,593; Viminol; Volazocine; Zenazocine; α-Chlorocodide; α-Chloromorphide; α-hydrocodol; α-Methylacetylfentanyl; α-Methylfentanyl; α-Methylthiofentanyl; β-Chlorocodide; β-hydroxyfentanyl; β-hydroxy thiofentanyl; β-Methylfentanyl; ψ-Akuammigine; derivatives thereof; and combinations thereof.

Examples of antimicrobial agents include, but are not limited to, alexidine, asphodelin A, atromentin, auranthine, austrocortilutein, austrocortirubin, azerizin, chlorbisan, chloroxine, cidex, cinoxacin, citreorosein, copper usnate, cupiennin, curvularin, DBNPA, dehydrocurvularin, desoxyfructo-serotonin, dichloroisocyanuric acid, elaiomycin, holtfreter's solution, malettinin, naphthomycin, neutrolin, niphimycin, nitrocefin, oxadiazoles, paenibacterin, proclin, ritiometan, ritipenem, silicone quaternary amine, stylisin, taurolidine, tirandamycin, trichloroisocyanuric acid, triclocarban, and combinations thereof.

Examples of antibacterials include, but are not limited to, acetoxycycloheximide, aciduliprofundum, actaplanin, actinorhodin, alazopeptin, albomycin, allicin, allistatin, allyl isothiocyanate, ambazone, aminocoumarin, aminoglycosides, 4-aminosalicylic acid, ampicillin, ansamycin, anthramycin, antimycin A, aphidicolin, aplasmomycin, archaeocin, arenicin, arsphenamine, arylomycin A2, ascofuranone, aspergillic acid, avenanthramide, avibactam, azelaic acid, bafilomycin, bambermycin, beauvericin, benzoyl peroxide, blasticidin S, bottromycin, brilacidin, caprazamycin, carbomycin, cathelicidin, cephalosporins, ceragenin, chartreusin, chromomycin A3, citromycin, clindamycin, clofazimine, clofoctol, clorobiocin, coprinol, coumermycin A1, cyclic lipopeptides, cycloheximide, cycloserine, dalfopristin, dapsone, daptomycin, debromomarinone, 17-dimethylaminoethylamino-17-demethoxy geldanamycin, echinomycin, endiandric acid C, enediyne, enviomycin, eravacycline, erythromycin, esperamicin, etamycin, ethambutol, ethionamide, (6S)-6-fluoroshikimic acid, fosfomycin, fosmidomycin, friulimicin, furazolidone, furonazide, fusidic acid, geldanamycin, gentamycin, gepotidacin, glycyclclines, glycyrrhizol, gramicidin S, guanacastepene A, hachimycin, halocyamine, hedamycin, helquinoline, herbimycin, hexamethylenetetramine, hitachimycin, hydramacin-1, isoniazid, kanamycin, katanosin, kedarcidin, kendomycin, kettapeptin, kidamycin, lactivicin, lactocillin, landomycin, landomycinone, lasalocid, lenapenem, leptomycin, lincosamides, linopristin, lipiarmycins, macbecin, macrolides, macromomycin B, maduropeptin, mannopeptimycin glycopeptide, marinone, meclocycline, melafix, methylenomycin A, methylenomycin B, monensin, moromycin, mupirocin, mycosubtilin, myriocin, myxopyronin, naphthomycin A, narasin, neocarzinostatin, neopluramycin, neosalvarsan, neothramycin, netropsin, nifuroxazide, nifurquinazol, nigericin, nitrofural, nitrofurantoin, nocathiacin I, novobiocin, omadacycline, oxacephem, oxazolidinones, penicillins, peptaibol, phytoalexin, plantazolicin, platensimycin, plectasin, pluramycin A, polymixins, polyoxins, pristinamycin, pristinamycin IA, promin, prothionamide, pulvinone, puromycin, pyocyanase, pyocyanin, pyrenocine, questiomycin A, quinolones, quinupristin, ramoplanin, raphanin, resistome, reuterin, rifalazil, rifamycins, ristocetin, roseophilin, salinomycin, salinosporamide A, saptomycin, saquayamycin, seraticin, sideromycin, sodium sulfacetamide, solasulfone, solithromycin, sparassol, spectinomycin, staurosporine, streptazolin, streptogramin, streptogramin B, streptolydigin, streptonigrin, styelin A, sulfonamides, surfactin, surotomycin, tachyplesin, taksta, tanespimycin, telavancin, tetracyclines, thioacetazone, thiocarlide, thiolutin, thiostrepton, tobramycin, trichostatin A, triclosan, trimethoprim, trimethoprim, tunicamycin, tyrocidine, urauchimycin, validamycin, viridicatumtoxin B, vulgamycin, xanthomycin A, xibornol, amikacin, amoxicillin, ampicillin, atovaquone, azithromycin, aztreonam, bacitracin, carbenicillin, cefadroxil, cefazolin, cefdinir, cefditoren, cefepime, cefiderocol, cefoperazone, cefotetan, cefoxitin, cefotaxime, cefpodoxime, cefprozil, ceftaroline, ceftazidime, ceftibuten, ceftizoxime, ceftriaxone, chloramphenicol, colistimethate, cefuroxime, cephalexin, cephradine, cilastatin, cinoxacin, ciprofloxacin, clarithromycin, clindamycin, dalbavancin, dalfopristin, daptomycin, demeclocycline, dicloxacillin, doripenem, doxycycline, eravacycline, ertapenem, erythromycin, fidaxomicin, fosfomycin, gatifloxacin, gemifloxacin, gentamicin, imipenem, lefamulin, lincomycin, linezolid, lomefloxacin, loracarbef, meropenem, metronidazole, minocycline, moxifloxacin, nafcillin, nalidixic acid, neomycin, norfloxacin, ofloxacin, omadacycline, oritavancin, oxacillin, oxytetracycline, paromomycin, penicillin, pentamidine, piperacillin, plazomicin, quinupristin, rifaximin, sarecycline, secnidazole, sparfloxacin, spectinomycin, sulfamethoxazole, sulfisoxazole, tedizolid, telavancin, telithromycin, ticarcillin, tigecycline, tobramycin, trimethoprim, trovafloxacin, vancomycin, and combinations thereof.

Examples of antifungals include, but are not limited to, abafungin, acibenzolar, acibenzolar-S-methyl, acrisorcin, allicin, aminocandin, amorolfine, amphotericin B, anidulafungin, azoxystrobin, bacillomycin, bacillus pumilus, barium borate, benomyl, binapacryl, boric acid, bromine monochloride, bromochlorosalicylanilide, bupirimate, butenafine, candicidin, caprylic acid, captafol, captan, carbendazim, caspofungin, cerulenin, chloranil, chlormidazole, chlorophetanol, chlorothalonil, chloroxylenol, chromated copper arsenate, ciclopirox, cilofungin, cinnamaldehyde, clioquinol, copper(I) cyanide, copper(II) arsenate, cruentaren, cycloheximide, davicil, dehydroacetic acid, dicarboximide fungicides, dichlofluanid, dimazole, diphenylamine, echinocandin, echinocandin B, epoxiconazole, ethonam, falcarindiol, falcarinol, famoxadone, fenamidone, fenarimol, fenpropimorph, fentin acetate, fenticlor, filipin, fluazinam, fluopicolide, flusilazole, fluxapyroxad, fuberidazole, griseofulvin, halicylindramide, haloprogin, hamycin, hexachlorobenzene, hexachlorocyclohexa-2,5-dien-1-one, 5-hydroxy-2(5H)-furanone, iprodione, lime sulfur, mancozeb, maneb, melafix, metalaxyl, metam sodium, methylisothiazolone, methylparaben, micafungin, miltefosine, monosodium methyl arsenate, mycobacillin, myclobutanil, natamycin, beta-nitrostyrene, nystatin, paclobutrazol, papulacandin B, parietin, pecilocin, pencycuron, pentamidine, pentachloronitrobenzene, pentachlorophenol, perimycin, 2-phenylphenol, polyene antimycotic, propamocarb, propiconazole, pterulone, ptilomycalin A, pyrazophos, pyrimethanil, pyrrolnitrin, selenium disulfide, sparassol, strobilurin, sulbentine, tavaborole, tebuconazole, terbinafine, theonellamide F, thymol, tiabendazole, ticlatone, tolciclate, tolnaftate, triadimefon, triamiphos, tribromometacresol, 2,4,6-tribromophenol, tributyltin oxide, triclocarban, triclosan, tridemorph, trimetrexate, undecylenic acid, validamycin, venturicidin, vinclozolin, vinyldithiin, vusion, xanthene, zinc borate, zinc pyrithione, zineb, ziram, voriconazole, itraconazole, posaconazole, fluconazole, ketoconazole, clotrimazole, isavuconazonium, miconazole, caspofungin, anidulafungin, micafungin, griseofulvin, terbinafine, flucytosine, terbinafine, nystatin, amphotericin b., and combinations thereof.

Examples of antivirals include, but are not limited to, afovirsen, alisporivir, angustific acid, angustifodilactone, alovudine, beclabuvir, 2,3-bis(acetylmercaptomethyl)quinoxaline, brincidofovir, dasabuvir, docosanol, fialuridine, ibacitabine, imiquimod, inosine, inosine pranobex, interferon, metisazone, miltefosine, neokadsuranin, neotripterifordin, ombitasvir, oragen, oseltamivir, pegylated interferon, podophyllotoxin, radalbuvir, semapimod, tecovirimat, telbivudine, theaflavin, tilorone, triptofordin C-2, variecolol, Zmapp, abacavir, acyclovir, adefovir, amantadine, amprenavir, atazanavir, balavir, baloxavir marboxil, boceprevir, cidofovir, cobicistat, daclatasvir, darunavir, delavirdine, didanosine, docasanol, dolutegravir, doravirine, ecoliever, edoxudine, efavirenz, elvitegravir, emtricitabine, enfuvirtide, entecavir, etravirine, famciclovir, fomivirsen, fosamprenavir, forscarnet, fosnonet, famciclovir, favipravir, fomivirsen, foscavir, ganciclovir, ibacitabine, idoxuridine, indinavir, inosine, inosine pranobex, interferon type I, interferon type II, interferon type III, lamivudine, letermovir, letermovir, lopinavir, loviride, maraviroc, methisazone, moroxydine, nelfinavir, nevirapine, nitazoxanide, oseltamivir, peginterferon alfa-2a, peginterferon alfa-2b, penciclovir, peramivir, pleconaril, podophyllotoxin, pyramidine, raltegravir, remdesevir, ribavirin, rilpivirine, rimantadine, rintatolimod, ritonavir, saquinavir, simeprevir, sofosbuvir, stavudine, tarabivirin, telaprevir, telbivudine, tenofovir alafenamide, tenofovir disoproxil, tenofovir, tipranavir, trifluridine, trizivir, tromantadine, umifenovir, valaciclovir, valganciclovir, vidarabine, zalcitabine, zanamivir, zidovudine. And combinations thereof.

### Methods

Additionally disclosed herein are methods of using the abovementioned devices to treat or prevent an aneurysm by disposing the device on an outer surface of a blood vessel of a subject, termed “exografting.” As used herein “blood vessel” can refer to an entire length of a blood vessel or to a portion of a blood vessel, such as a segment of a blood vessel. For example, various aspects include disposing the device on the outer surface of an aortic blood vessel, such as an ascending aorta, thoracic aorta, abdominal aorta, or branches of the aortic arch. In some aspects, the abovementioned device can be deployed in patients undergoing concomitant procedures.

Additionally, the method can further include fastening the device such that the biomatrix core contacts the adventitia of the blood vessel of the subject. The device may be fastened by, for example, a zip and lock fastening mechanism, or by any of the other fastening mechanisms discussed above. The device can be fastened around the blood vessel to sufficiently restrict an expanding dilation of the vessel, while still being compliant to allow free flow of blood through the vessel.

In various aspects, the exografting method includes disposing a device including an outer scaffold having a metal framework. For example, the metal framework can include a biocompatible material such as stainless steel, titanium alloys, tantalum alloys, nickel alloys such as nickel-chromium alloys, cobalt alloys such as cobalt-chromium alloys and precious metals. Shape memory alloys such as the nickel-titanium alloy, Nitinol® can be used. In some aspects, the metal framework comprises Nitinol® and/or stainless steel. In various aspects, the outer scaffold maintains the size of the aorta over an extended time period.

Also discussed herein are methods for supporting a blood vessel by disposing the device on an outer surface of the blood vessel. For example, various aspects include disposing the device on the outer surface of an aortic blood vessel, such as an ascending aorta.

In various aspects, the method includes disposing a device including an outer scaffold having a metal framework. For example, the metal framework can include a biocompatible material such as stainless steel, titanium alloys, tantalum alloys, nickel alloys such as nickel-chromium alloys, cobalt alloys such as cobalt-chromium alloys and precious metals. Shape memory alloys such as the nickel-titanium alloy, Nitinol® can be used. In some aspects, the metal framework comprises Nitinol® and/or stainless steel. In various aspects, the outer scaffold maintains the size of the aorta over an extended time period.

The exografting method can be used to support a blood vessel in subjects with various conditions. For example, the method can be used to support a blood vessel in a subject who is not a candidate for aortic replacement. Additionally, in some aspects, the method can be used to support a blood vessel in a subject having a connective tissue disorder, such as Marfan's Syndrome, Loeys-Dietz Syndrome, Familial Aortic Aneurysm, Bicuspid Aortic Valve with Aortic Dilation, Mitral Valve Prolapse Syndrome, Marfan Habitus, Congenital Contractural Arachnodactyly (Beals Syndrome), Sjögren's syndrome, Stickler syndrome, Shprintzen-Goldberg syndrome, Weill-Marchesani syndrome, and Ehlers-Danlos syndrome. In some cases, the connective tissue disorder is Marfan's Syndrome.

Other advantages of the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

### Examples

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Bioengineered Scaffolds for Exografting

The device 100 shown in FIGS. 1-2 depicts one embodiment of the bioengineered scaffold. The device 100 has an inner core 110 comprising a biomatrix and an outer scaffold 120 comprising a biocompatible polymer. The biomatrix is configured to allow migration of fibroblast through the biomatrix, thereby. Thus, when the device 100 is attached to a blood vessel, The device is configured to be secured around a blood vessel using a fastening mechanism 130 comprising a zip 130a and lock 130b.

Although FIGS. 1-2 show a device having a type of zip and lock fastening mechanism, various other fastening mechanisms can be utilized, such as threads, screws, hooks, zips, fasteners, clips, flaps, claspers, springs, claspers, staplers, grips, zippers, hooks and corresponding eyes, hook and loop reclosable fastener squares, hook and loop reclosable fastener strips, hook and loop reclosable fastener dots, hooks-and-loops, e.g., Velcro™-type fasteners, straps, holes and string, sutures, wires, cables, tabs, poppers, nails, buttons and corresponding button holes, press buttons brackets, glues, adhesives, or any combination thereof.

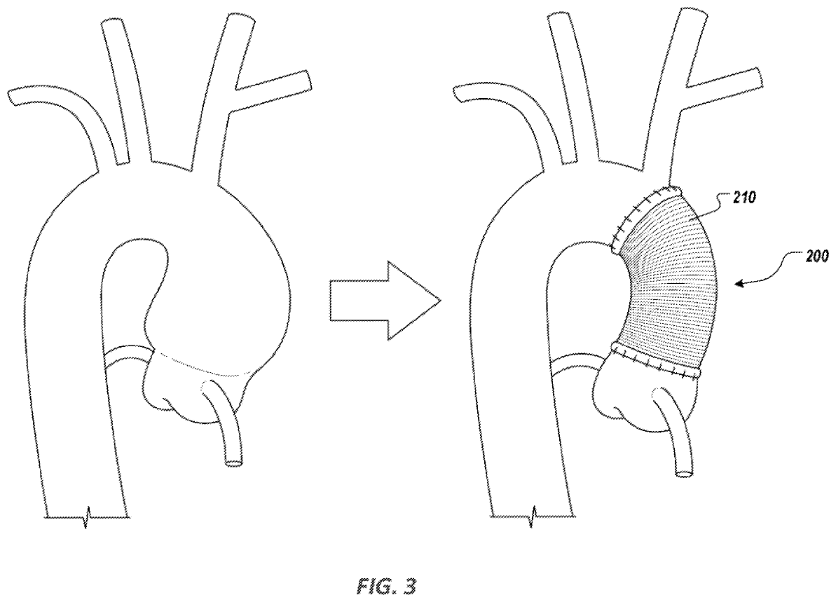

FIG. 3 depicts a device 200 disposed on the adventitia of the ascending aorta to restrict vessel dilation according to another aspect. The device 200 includes an outer scaffold 220 capable of expanding and/or collapsing. The outer scaffold 220 of the device 200 is collapsible and expandable using a pattern of repeating rounded units which can be axially collapsed to a compacted device to facilitate insertion through the incision.

The construction and arrangement of the device as shown in the exemplary embodiment is illustrative only. Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A device comprising:
an inner core comprising a biomatrix; and
an outer scaffold comprising a biocompatible polymer.

2. The device of claim 1, wherein the biocompatible polymer comprises polyetheretherketone (PEEK) or polyetherketoneketone (PEKK).

3. The device of any one of claims 1-2, wherein the outer scaffold further comprises a metal framework.

4. The device of claim 3, wherein the metal framework comprises nitinol or stainless steel.

5. The device of any one of claims 1-4, wherein the device comprises a fastening mechanism configured to secure placement of the device on an outer surface of a blood vessel.

6. The device of claim 5, wherein the fastening mechanism comprises a zip and a lock.

7. The device of any one of claims 1-6, wherein the biomatrix is configured to allow migration of fibroblasts through the biomatrix.

8. The device of any one of claims 1-7, wherein the biomatrix is collagen-enriched.

9. A method of supporting a blood vessel, comprising:
disposing the device of any one of claims 1-8 on an outer surface of a blood vessel of a subject.

10. The method of claim 9, wherein the blood vessel is an aortic blood vessel.

11. The method of claim 10, wherein the aortic blood vessel is an ascending aorta.

12. The method of any one of claims 8-11, wherein the outer scaffold maintains the size of the aorta over an extended time period.

13. The method of any one of claims 8-12, wherein the subject is an elderly subject.

14. The method of any one of claims 8-13, wherein the subject is not a candidate for aortic replacement.

15. The method of any one of claims 8-14, wherein the subject suffers from a connective tissue disorder.

16. The method of claim 15, wherein the connective tissue disorder is Marfan syndrome.

17. The method of any one of claims 8-16, further comprising fastening the device such that the biomatrix core contacts the adventitia of the blood vessel.

18. A method of preventing or treating an aneurysm, comprising:
disposing the device of any one of claims 1-8 on an outer surface of a blood vessel of a subject.

19. The method of claim 18, wherein the blood vessel is an aortic blood vessel.

20. The method of claim 19, wherein the aortic blood vessel is an ascending aorta.

21. The method of any one of claims 18-20, further comprising fastening the device such that the biomatrix core contacts the adventitia of the blood vessel.
